(19) United States
(12) Patent Application Publication
(10) Pub. No.: US 2012/0323145 A1
(43) Pub. Date: Dec. 20, 2012

(54) GUIDEWIRE

(75) Inventors: Satoshi NAGANO, Nagoya-shi (JP); Hideaki MAKI, Nagoya-shi (JP); Yuuya KANAZAWA, Nagoya-shi (JP)

(73) Assignee: ASAHI INTECC CO., LTD., Nagoya-shi (JP)

(21) Appl. No.: 13/487,756

(22) Filed: Jun. 4, 2012

(30) Foreign Application Priority Data
Jun. 15, 2011 (JP) 2011-133051

(51) Int. Cl. A61M 25/09

(52) U.S. Cl. 600/585

(57) ABSTRACT

A guidewire includes a core shaft having front and rear sections including a front end portion and a rear section, respectively, an outer coil body that covers the front section, and an inner coil body that is disposed within the outer coil body between the outer coil body and the core shaft, and covers the front section. The outer coil body includes a cylindrical large-diameter section positioned adjacent the rear end portion, a cylindrical small-diameter section positioned adjacent the front end portion, and a tapered section that connects the cylindrical large-diameter section to the cylindrical small-diameter section with a connecting portion provided therebetween, the tapered section having a diameter that decreases from an end adjacent the rear end portion to an end adjacent the front end portion. The inner coil body is disposed at least in the tapered section.

### CROSS REFERENCE TO RELATED APPLICATIONS

This application claims priority to Japanese Patent Application No. 2011-133051 filed with the Japan Patent Office on Jun. 15, 2011, the entire contents of which are hereby incorporated by reference.

### BACKGROUND

The disclosed embodiments relate to a medical device. More specifically, the disclosed embodiments relate to a guidewire.

Guidewires are medical mechanical devices used to guide a device such as a balloon or a stent to a lesion in percutaneous transluminal coronary angioplasty (PTCA).

U.S. Pat. No. 5,345,945 discloses an example of such a guidewire which includes a core shaft and a coil body. The core shaft includes a small-diameter front section and a large-diameter rear section. The coil body is wound around the outer periphery of the front section and includes a tapered section having a diameter that decreases toward a front end portion of the front section. The front section and the rear section of the core shaft correspond to a distal portion and a proximal portion, respectively, of the guidewire. The distal portion of the guidewire is inserted into the body, and the proximal portion of the guidewire is operated by an operator, such as a doctor.

Since the guidewire according to the related art described in U.S. Pat. No. 5,345,945 includes the coil body including the tapered section that has a diameter that decreases toward the front end portion, the guidewire has high performance of penetration into a lesion

### SUMMARY

However, when existing guidewires, such as the guidewire in the above-discussed related art are rotated, pushed, or pulled after being advanced into the lesion, their coil bodies are easily deformed around the tapered section. This tendency increases, in particular, when the diameter of the distal portion of the guidewire is reduced, that is, when the diameter of the coil body is reduced, to improve the performance of penetration into a lesion.

The inventors of the present invention have studied the cause of the deformation of the coil body, and have determined that said deformation results from the shape of the tapered section, which gradually changes from a large-diameter area to a small-diameter area, and therefore an applied external force, such as torsion and bending force, more easily concentrates at the tapered section compared to cylindrical sections having a uniform diameter.

As a result of further studies conducted by the inventors based on the above findings, the present inventors have determined that the deformation can be prevented by reinforcing the tapered section by arranging another coil body in the tapered section which itself is easily deformed. Thus, the guidewire according to the embodiments of the present invention has been completed.

According to an aspect of the present invention, a guidewire includes a core shaft including a front section including a front end portion and a rear section including a rear end portion, an outer coil body that covers the front section, and an inner coil body that is disposed within the outer coil body and covers the front section. The outer coil body includes a cylindrical large-diameter section positioned near the rear end portion, a cylindrical small-diameter section positioned near the front end portion, and a tapered section that connects the large-diameter section to the small-diameter section with a connecting portion provided therebetween. The tapered section has a diameter that decreases from an end near the rear end portion to an end near the front end portion. Of the large-diameter section, the small-diameter section, the connecting portion, and the tapered section, the inner coil body is disposed at least in the tapered section,

### BRIEF DESCRIPTION OF THE DRAWINGS

FIG. 1 is a schematic sectional view of a guidewire according to a first embodiment of the present invention taken along a longitudinal direction of the guidewire.

FIG. 2 is an enlarged sectional view of an area around a distal portion of the guidewire illustrated in FIG. 1.

FIG. 3 is an enlarged sectional view of an area around a distal portion of a guidewire according to a second embodiment of the present invention.

FIG. 4 is an enlarged sectional view of an area around a distal portion of a guidewire according to a third embodiment of the present invention.

FIG. 5 is an enlarged sectional view of an area around a distal portion of a guidewire according to a fourth embodiment of the present invention.

### DETAILED DESCRIPTION OF EMBODIMENTS

A guidewire according to a first embodiment of the present invention will now be described below with reference to the drawings.

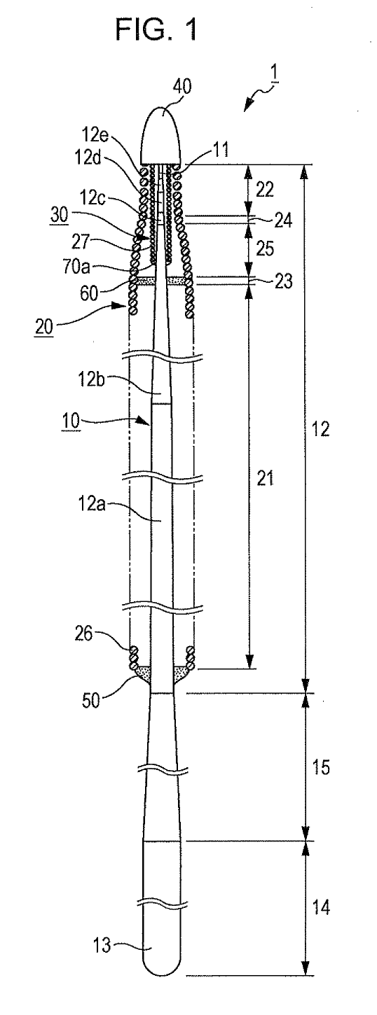

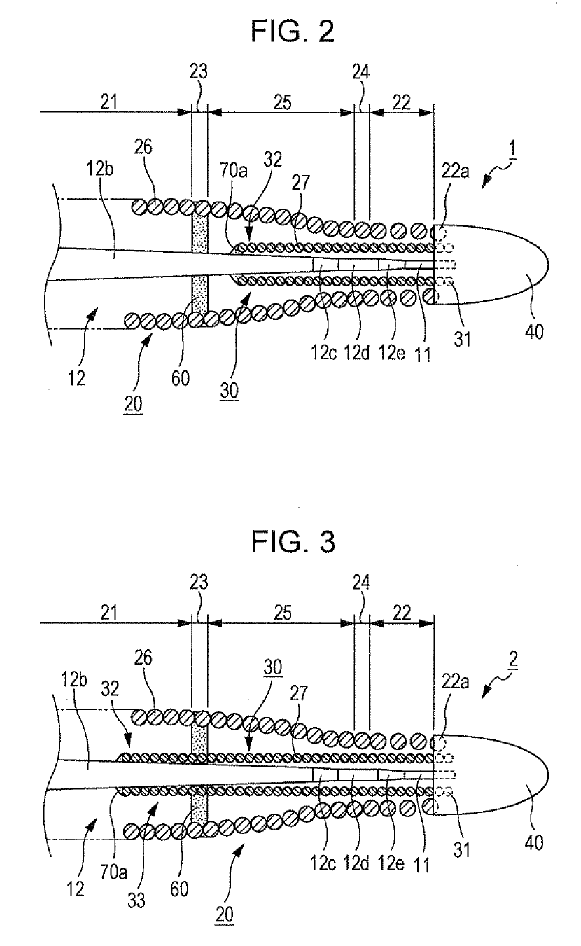

FIG. 1 is a schematic sectional view of a guidewire 1 according to a first embodiment of the present invention taken along a longitudinal direction of the guidewire 1. FIG. 2 is an enlarged sectional view of an area around a distal portion of the guidewire 1 illustrated in FIG. 1. In the following description, a distal portion of the guidewire 1 and a front section of a core shaft 10 are denoted by the same reference numeral 12, and a proximal portion of the guidewire 1 and a rear section of the core shaft 10 are denoted by the same reference numeral 14. In addition, a portion of an outer coil body 20 may be shown by broken lines, and detailed illustration of the portion of the outer coil body 20 may thus be omitted.

The guidewire 1 according to the embodiment illustrated in FIG. 1 includes the core shaft 10, the outer coil body 20, and an inner coil body 30. The core shaft 10 includes the front section 12, which includes a front end portion 11, and the rear section 14, which includes a rear end portion 13. The outer coil body 20 covers the front section 12. The inner coil body 30 is disposed within the outer coil body 20 (i.e., radially between the outer coil body 20 and the core shaft 10), and the inner coil body 30 covers the front section 12 of the core shaft 10. The detailed structure of the guidewire 1 will now be described.

The core shaft 10 includes the front section 12, which includes the front end portion 11 and has a small diameter, a tapered intermediate section 15, which is connected to the front section 12, and the rear section 14, which is connected to the intermediate section 15 and includes the rear end portion 13. The rear section 14 has a larger diameter than the intermediate section 15 and the front section 12.

As illustrated in FIGS. 1 and 2, the front section 12 includes, in order from the end near the rear end portion 13 to the front end portion 11, a first small-diameter portion 12a, a first tapered portion 12b, a second tapered portion 12c, a second small-diameter portion 12d, a third tapered portion 12e, and the front end portion 11, all of which are connected to each other. The small-diameter portions are columnar portions having a substantially uniform outer diameter, and the tapered portions are portions having a diameter that gradually decreases along the direction from the rear end portion 13 to the front end portion 11. As a whole, the front section 12 has a diameter that decreases stepwise from the end near the rear end portion 13 to the front end portion 11. Accordingly, the rigidity of the front section 12 gradually decreases and the flexibility thereof gradually increases toward the front end portion 11.

The outer coil body 20 is formed by helically winding a single wire 26, and has a tubular shape with a through hole therein. With regard to the detailed shape of the outer coil body 20, the outer coil body 20 includes a large-diameter section 21 positioned at the rear end of the outer coil body 20, a tapered section 25 connected to the large-diameter section 21 with a first connecting portion 23 provided therebetween, and a small-diameter section 22 positioned at the front end of the outer coil body 20 and connected to the tapered section 25 with a second connecting portion 24 provided therebetween.

The diameter of the tapered section 25 decreases from the end near the large-diameter section 21 to the end near the small-diameter section 22. With this arrangement, an applied external force, such as a bending force, more easily concentrates at the tapered section 25 than at the large-diameter and small-diameter sections 21 and 22 having a uniform diameter, and therefore the tapered section 25 is easily deformed. In addition, the first and second connecting portions 23 and 24 have a shape that suddenly changes, and therefore the applied external force particularly easily concentrates at the first and second connecting portions 23 and 24. Therefore, the first and second connecting portions 23 and 24 are very easily deformed. As used herein, each connecting portion corresponds to either several turns of wire in a range in which the diameter decreases from that of the cylindrical large-diameter section 21, which has a substantially uniform diameter from the end near the rear end portion to the end near the front end portion, to that of the tapered section 25 (first connecting portion); or several turns of wire in a range in which the diameter decreases from that of the tapered section 25, which has a diameter that decreases from the end near the rear end portion to the end near the front end portion, to that of the cylindrical small-diameter section 22, which has a substantially uniform diameter (second connecting portion). Although a section corresponding to a single turn of wire is illustrated as each connecting portion in the drawings, this is merely because the wire that forms the outer coil body 20 is drawn as if it has a larger diameter than the actual diameter for simplification of the drawings, and therefore the actual dimensions and structure are different from those illustrated in the drawings.

The front section 12 and the inner coil body 30 that partially covers the front section 12 are inserted in the outer coil body 20. The outer coil body 20 substantially entirely covers the front section 12 and the inner coil body 30.

In the small-diameter section 22, the wire 26 is loosely wound such that portions of the wire 26 that are adjacent to each other are spaced from each other. Therefore, the small-diameter section 22 is flexible. In the part of the outer coil body 20 other than the small-diameter section 22, the wire 26 is densely wound such that portions of the wire 26 that are adjacent to each other are in contact with each other. Therefore, this part of the outer coil body 20 is not easily twisted and is capable of efficiently transmitting a torque generated when the proximal portion 14 of the guidewire 1 is rotated to the front end portion of the distal portion 12 of the guidewire 1. The outer coil body 20 may instead be densely or loosely wound over the entire body thereof.

The inner coil body 30 is formed of a multiple-wire coil obtained by winding a plurality of wires 27. More specifically, the inner coil body 30 is formed by helically winding the wires 27, and has a tubular shape with a through hole therein. Therefore, plastic deformation of the inner coil body 30 does not easily occur compared to a single-wire coil obtained by winding a single wire. In addition, when a first end of the inner coil body 30 is rotated, a second end easily rotates so as to follow the first end. Thus, the inner coil body 30 has high rotation-following performance. The number of wires included in the inner coil body (multiple-wire coil) 30 is preferably 6 to 8.

Of the large-diameter section 21, the small-diameter section 22, the connecting portions 23 and 24, and the tapered section 25, the inner coil body 30 having the above-described structure is disposed at least in the tapered section 25. In the guidewire 1 illustrated in FIG. 1, the inner coil body 30 is disposed in the small-diameter section 22, the second connecting portion 24 that connects the small-diameter section 22 to the tapered section 25, and the tapered section 25.

However, as described in the following embodiments, the inner coil body 30 may instead be disposed so as to extend in the large-diameter section 21, the small-diameter section 22, the connecting portions 23 and 24, and the entire body of the tapered section 25.

A part of the front section 12, more specifically, a part of the first tapered portion 12b, the second tapered portion 12c, the second small-diameter portion 12d, the third tapered portion 12e, and the front end portion 11, is inserted in the inner coil body 30. Thus, the inner coil body 30 partially covers the front section 12.

The front end portion 11 of the core shaft 10, a front end portion 22a of the small-diameter section 22, and a front end portion 31 of the inner coil body 30 are fixed to each other by a front tip portion 40.

The front tip portion 40 has a conical shape, and the vertex of the cone serves as a front end portion of the guidewire 1. The front tip portion 40 is made of a solder material containing Au. The front tip portion 40 is preferably made of Sn—Au alloy.

A rear end portion of the first small-diameter portion 12a and a rear end portion of the large-diameter section 21 of the outer coil body 20 illustrated in FIG. 1 are fixed to each other by a rear-end solder part 50.

A front end portion of the large-diameter section 21 of the outer coil body 20 (the first connecting portion 23 that connects the large-diameter section 21 to the tapered section 25) and the first tapered portion 12b are fixed to each other by an intermediate solder part 60. The outer coil body 20 and the front section 12 may be additionally fixed to each other by one or more solder parts other than the rear-end solder part 50 and the intermediate solder part 60 at arbitrary positions.

A rear end portion 32 of the inner coil body 30 and the first tapered portion 12b are fixed to each other by an inner rear-end solder part 70a at a position closer to the front end portion 11 of the core shaft 10 than the intermediate solder part 60. The inner coil body 30 and the front section 12 (the first tapered portion 12b) may be additionally fixed to each other by one or more solder parts other than the inner rear-end solder part 70a at arbitrary positions.

The guidewire according to the FIG. 1 embodiment may be manufactured by, for example, the following method. First, the core shaft 10 is produced by forming a core wire into the above-described shape by, for example, a taper cutting process or a pressing process. Then, the front end section 12 of the core shaft 10 is inserted into the inner coil body 30 and soldered to the inner coil body 30 at a predetermined position. Then, the front end section 12 of the core shaft 10 and the inner coil body 30 are inserted into the outer coil body 20 and soldered to the outer coil body 20 at predetermined positions. Thus, the guidewire according to the FIG. 1 embodiment is manufactured.

The effects of the guidewire according to the FIG. 1 embodiment include at least the following effects:

(1) In the guidewire according to the FIG. 1 embodiment, the inner coil body 30 is disposed in the tapered section 25, which itself is easily deformed since the applied external force, such as bending force and torsion, easily concentrates thereat. Thus, the tapered section 25 is reinforced. Accordingly, even when the guidewire is manually operated, the outer coil body 20 including the tapered section 25 is not easily deformed. As a result, the outer coil body 20 is not easily damaged.

(2) The inner coil body 30 is disposed in the tapered section 25, and a part of the guidewire including the tapered section 25 has a double coil structure. Therefore, a torque generated when the proximal portion of the guidewire is rotated is efficiently transmitted to the front end portion of the distal portion of the guidewire through the outer coil body 20 and the inner coil body 30. Thus, the guidewire has high torque-transmitting performance.

(3) The guidewire includes the outer coil body 20, including the tapered section 25, having a diameter that decreases from the end near the rear end portion 13 of the core shaft 10 to the end near the front end portion 11 of the core shaft 10, and the distal portion of the guidewire is shaped such that the diameter thereof decreases toward the tip. Accordingly, the guidewire has high performance of penetration into a lesion.

(4) The applied external force concentrates particularly at the connecting portions 23 and 24 that connect the tapered section 25 to the cylindrical large-diameter section 21 and the small-diameter section 22 since the connecting portions 23 and 24 have a shape that suddenly changes. However, in the guidewire according to the FIG. 1 embodiment, the inner coil body 30 is disposed not only in the tapered section 25 but also in one of the connecting portions (24). Therefore, deformation of the outer coil body 20 is further effectively prevented and the torque-transmitting performance can be improved.

(5) The small-diameter section 22 is particularly flexible compared to the large-diameter section 21, and the strength of the small-diameter 22 section may be low depending on the diameter thereof. However, in the guidewire according to the FIG. 1 embodiment, the inner coil body 30 is disposed in the tapered section 25, the small-diameter section 22, and the second connecting portion 24 that connects the tapered section 25 to the small-diameter section 22. Therefore, deformation of the outer coil body 20 can be further effectively prevented.

(6) The small-diameter section 22, which is located at the front end of the outer coil body 20, is originally flexible. In addition, since the small-diameter section 22 is advanced to a deep part of the lesion, the applied external force easily concentrates at the small-diameter section 22. Therefore, the small-diameter section 22 is particularly easily deformed. However, in the guidewire according to the present embodiment, since the inner coil body 30 is disposed in the small-diameter section 22, deformation of the small-diameter section 22 can be prevented.

(7) The front end portion of the core shaft 10, the front end portion of the small-diameter section 22 of the outer coil body 20, and the front end portion of the inner coil body 30 are fixed to each other by the front tip portion 40. Therefore, the torque applied to the rear section of the core shaft 10 can be efficiently transmitted to the front tip portion 40 through the outer coil body 20 and the inner coil body 30. Thus, the torque-transmitting performance can be further improved.

(8) The inner coil body 30 is formed of a multiple-wire coil obtained by winding a plurality of wires, and therefore the inner coil body 30 is not easily plastically deformed and has high rotation-following performance. As a result, at least the above-described effects (1), (2), and (4) to (7) can be enhanced.

(9) Since the front tip portion 40 has a conical shape, the ability to penetrate into a lesion can be improved.

(10) The front tip portion 40 is made of a solder material containing Au. Therefore, the front tip portion 40 has a higher strength than that of a front tip portion made of, for example, a solder material containing Ag—Sn alloy, and does not easily break even when the length thereof is reduced.

A guidewire according to a second embodiment of the present invention will now be described with reference to the drawings. The guidewire according to the second embodiment has a structure similar to that of the above-described guidewire according to the first embodiment except that the inner coil body 30 is disposed in the large-diameter section 21, the first connecting portion 23, the tapered section 25, the second connecting portion 24, and the small-diameter section 22. Therefore, explanations of features similar to those of the guidewire according to the first embodiment will be omitted.

FIG. 3 is an enlarged sectional view of an area around a distal portion of a guidewire 2 according to the second embodiment of the present invention.

Referring to FIG. 3, in the guidewire 2 according to the second embodiment, a part of the first tapered portion 12b, the second tapered portion 12e, the second small-diameter portion 12d, the third tapered portion 12e, and the front end portion 11 are inserted in the inner coil body 30, and the inner coil body 30 is disposed in the large-diameter section 21, the first connecting portion 23, the tapered section 25, the second connecting portion 24, and the small-diameter section 22.

The front end portion of the large-diameter section 21 of the outer coil body 20 (the first connecting portion 23 that connects the large-diameter section 21 to the tapered section 25), an intermediate portion 33 of the inner coil body 30, and the first tapered portion 12b are fixed to each other by the intermediate solder part 60.

The rear end portion 32 of the inner coil body 30 and the first tapered portion 12b are fixed to each other by the inner rear-end solder part 70a at a position closer to the rear end portion 13 of the core shaft 10 than the intermediate solder part 60.

The guidewire according to the second embodiment is manufactured by a method similar to the manufacturing method of the guidewire according to the first embodiment except that a longer inner coil body 30 is used.

The effects of the guidewire according to the second embodiment will now be described. The above-described effects (1) to (10) of the first embodiment can also be achieved by the guidewire according to the second embodiment. Further, at least the following effects (11) and (12) can be additionally achieved.

(11) The applied external force concentrates particularly at the first and second connecting portions (23, 24) since the first and second connecting portions (23, 24) have a shape that suddenly changes. However, in the guidewire according to the second embodiment, the inner coil body 30 is disposed not only in the tapered section 25 but also in the first and second connecting portions (23, 24). Therefore, deformation of the first and second connecting portions (23, 24) and the tapered section 25 is further effectively prevented and the torque-transmitting performance can be further improved.

(12) The first connecting portion 23, the intermediate portion 33 of the inner coil body 30, and the first tapered portion 12b are fixed to each other by the intermediate solder part 60. Thus, the outer coil body 20 and the inner coil body 30 are more strongly fixed to each other. As a result, the torque-transmitting performance can be further improved.

A guidewire according to a third embodiment of the present invention will now be described with reference to the drawings. The guidewire according to the third embodiment has a structure similar to that of the above-described guidewire according to the first embodiment except that the inner coil body 30 is disposed in the first connecting portion 23, the tapered section 25, and the second connecting portion 24, but is not disposed in the large-diameter section 21 or the small-diameter section 22. Therefore, explanations of features similar to those of the guidewire according to the first embodiment will be omitted.

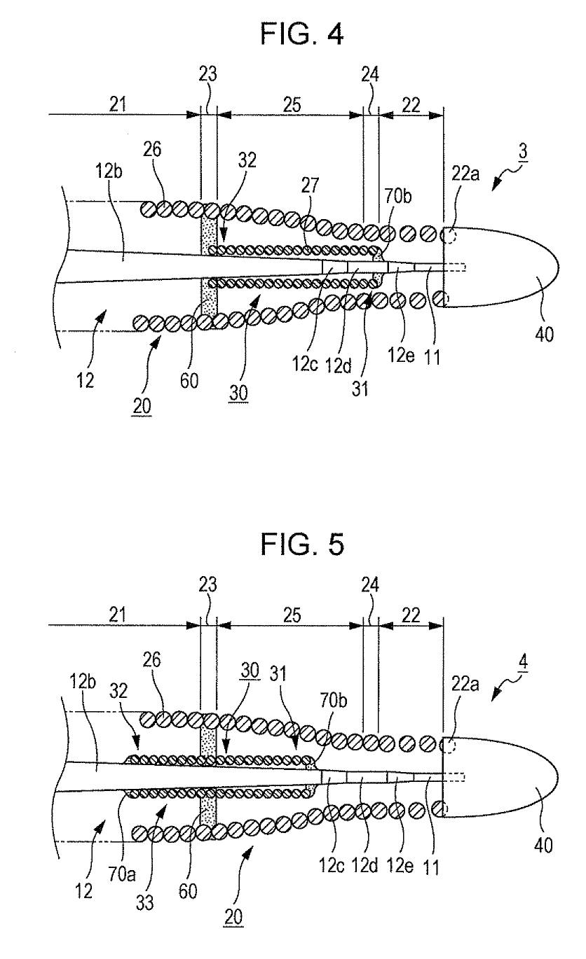

FIG. 4 is an enlarged sectional view of an area around a distal portion of a guidewire 3 according to the third embodiment of the present invention.

Referring to FIG. 4, in the guidewire 3 according to the third embodiment, a part of the first tapered portion 12b, the second tapered portion 12c, and a part of the second small-diameter portion 12d are inserted in the inner coil body 30, and the inner coil body 30 is disposed in the first connecting portion 23, the tapered section 25, and the second connecting portion 24.

The first connecting portion 23, the rear end portion 32 of the inner coil body 30, and the first tapered portion 12b are fixed to each other by the intermediate solder part (inner rear-end solder part) 60.

The front end portion 31 of the inner coil body 30 and the second small-diameter portion 12d are fixed to each other by an inner front-end solder part 70b.

The guidewire according to the third embodiment is manufactured by a method similar to the manufacturing method of the guidewire according to the first embodiment except that a shorter inner coil body 30 is used and the soldering positions are changed.

The effects of the guidewire according to the third embodiment will now be described. The above-described effects (1) to (4) and (8) to (12) can also be achieved by the guidewire according to the third embodiment. Further, at least the following effect (13) can be additionally achieved.

(13) Since the inner coil body 30 is not disposed in the small-diameter section 22, the distal portion of the guidewire is particularly flexible in the area around the front end thereof (area corresponding to the small-diameter section 22). Therefore, a part of the guidewire in the area around the front end thereof can be easily bent at a predetermined angle (shaped) in advance. When the guidewire is shaped in advance, vascular selectivity for thin blood vessels can be increased and the guidewire can be precisely maneuvered in the lesion. Thus, a guidewire having a high maneuverability can be provided.

A guidewire according to a fourth embodiment of the present invention will now be described with reference to the drawings. The guidewire according to the fourth embodiment has a structure similar to that of the above-described guidewire according to the first embodiment except that the inner coil body 30 is disposed in the large-diameter section 21, the first connecting portion 23, and the tapered section 25, but is not disposed in the second connecting portion 24 or the small-diameter section 22. Therefore, explanations of features similar to those of the guidewire according to the first embodiment will be omitted.

FIG. 5 is an enlarged sectional view of an area around a distal portion of a guidewire 4 according to the fourth embodiment of the present invention.

Referring to FIG. 5, in the guidewire 4 according to the fourth embodiment, a part of the first tapered portion 12b is inserted in the inner coil body 30, and the inner coil body 30 is disposed in the large-diameter section 21, the first connecting portion 23, and the tapered section 25.

The rear end portion 32 of the inner coil body 30 and the first tapered portion 12b are fixed to each other by the inner rear-end solder part 70a.

The first connecting portion 23, the intermediate portion 33 of the inner coil body 30, and the first tapered portion 12b are fixed to each other by the intermediate solder part 60.

The front end portion 31 of the inner coil body 30 and the first tapered portion 12b are fixed to each other by the inner front-end solder part 70b.

The guidewire according to the fourth embodiment is manufactured by a method similar to the manufacturing method of the guidewire according to the first embodiment except that the soldering positions of the inner coil body 30 are changed.

The effects of the guidewire according to the fourth embodiment will now be described. The above-described effects (1) to (4), (8) to (10), (12), and (13) can also be achieved by the guidewire according to the fourth embodiment. Further, at least the following effect (14) can be additionally achieved.

(14) The rear end portion 32 of the inner coil body 30 is fixed to the first tapered portion 12b of the core shaft 10, which is near the rear section thereof, by the inner rear-end solder part 70a. Therefore, the torque applied to the rear section of the core shaft 10 can be efficiently transmitted to the front end portion. Thus, the torque-transmitting performance can be further improved.

In the guidewire according to embodiments of the present invention, as described above, the small-diameter section is preferably positioned at the front end of the outer coil body 20. However, the small-diameter section may instead be disposed at a position shifted rearward from the front end of the outer coil body by a predetermined distance.

In the guidewire according to the present invention, as described above, the outer coil body 20 may include a single set including the large-diameter section 21, the tapered section 25, and the small-diameter section 22. Alternatively, however, the outer coil body 20 may include two or more sets which each include a large-diameter section 21, a tapered section 25, and a small-diameter section 22. In the case where the outer coil body 20 includes two or more sets which each include a large-diameter section 21, a tapered section 25, and a small-diameter section 22, these sections are preferably arranged in such an order that the diameter of the outer coil body 20 decreases stepwise from the end near the rear end portion 13 of the core shaft 10 to the end near the front end portion 11 of the core shaft 10. For example, in the direction from the rear end portion 13 to the front end portion 11 of the core shaft 10, a large-diameter section 21, a tapered section 25, and a small-diameter section 22 of a first set may be arranged in that order, and then a large-diameter section 21, a tapered section 25, and a small-diameter section 22 of a second set may be arranged in that order.

Preferably, the outer diameter of the small-diameter section 22 in the first set is equal to or larger than the outer diameter of the large-diameter section 21 in the second set. In the case where two or more sets which each include a large-diameter section 21, a tapered section 25, and a small-diameter section 22 are provided, the dimension of the step between the large-diameter section 21 and the small-diameter section 22 can be reduced. Therefore, the guidewire can be more smoothly advanced into the lesion. In the case where two or more sets which each include a large-diameter section 21, a tapered section 25, and a small-diameter section 22 are provided, the inner coil body 30 may be disposed at least in the tapered section 25 of one of the sets.

In the guidewire according to embodiments of the present invention, the front tip portion 40 preferably has a conical shape to improve penetration into a lesion. Alternatively, however, the front tip portion 40 may have a hemispherical shape instead. In the case where the front tip portion 40 has a hemispherical shape, even when the guidewire has high push-in performance based on high rigidity of the core shaft 10, the risk that the guidewire will penetrate a blood vessel can be reduced.

In the guidewire according to embodiments of the present invention, the solder parts including the front-end solder part, the intermediate solder part 60, the rear-end solder part 50, the inner front-end solder part 70b, and the inner rear-end solder part 70a may be formed of, for example, aluminum alloy solder, silver solder, gold solder, zinc solder, Sn—Pb alloy solder, Sn—Au alloy solder, Pb—Ag alloy solder, or Sn—Ag alloy solder. Preferably, in particular, the solder parts are made of gold solder or Sn—Au alloy solder. In such a case, the strength of the solder parts can be increased.

In the guidewire according to embodiments of the present invention, the core shaft 10 may be made of, for example, a stainless steel, a superelastic alloy such as Ni—Ti alloy, a piano wire, or a tungsten wire. The stainless steel may be, for example, a martensitic stainless steel, a ferritic stainless steel, an austenitic stainless steel, an austenitic-ferritic duplex stainless steel, or a precipitation hardened stainless steel. Preferably, the core shaft 10 is made of an austenitic stainless steel. In particular, SUS304, SUS316, or SUS316L is preferably used.

In the guidewire according to embodiments of the present invention, the inner coil body 30 is preferably formed of a multiple-wire coil obtained by winding a plurality of wires. However, the inner coil body 30 may instead be formed of a single-wire coil obtained by winding a single wire. In the case where the inner coil body 30 is formed of a single-wire coil, the inner coil body 30 has a higher flexibility than that of the inner coil body 30 formed of a multiple-wire coil.

In the guidewire according to embodiments of the present invention, the inner coil body 30 is formed of a single multiple-wire coil. However, the inner coil body 30 may instead be formed by connecting a plurality of multiple-wire coils. In the case where the inner coil body 30 is formed by connecting a plurality of multiple-wire coils, the multiple-wire coils may have different diameters (inner and outer diameters). In this case, the multiple-wire coils are preferably arranged such that the diameter thereof gradually decreases from the end near the rear end portion 13 of the core shaft 10 to the end near the front end portion 11 of the core shaft 10. Accordingly, the flexibility gradually increases toward the front end portion 11 of the guidewire, and the performance of penetration into a lesion can be improved while ensuring the flexibility of the distal portion of the guidewire.

In the guidewire according to embodiments of the present invention, the wires that form the inner coil body 30 may be made of, for example, a stainless steel, a superelastic alloy such as Ni—Ti alloy, a piano wire, or a tungsten wire. The stainless steel may be, for example, a martensitic stainless steel, a ferritic stainless steel, an austenitic stainless steel, an austenitic-ferritic duplex stainless steel, or a precipitation hardened stainless steel. Preferably, the wires are made of an austenitic stainless steel. In particular, SUS304, SUS316, or SUS316L is preferably used.

In the guidewire according to embodiments of the present invention, the wire that forms the outer coil body 20 may be made of, for example, a stainless steel, such as a martensitic stainless steel, a ferritic stainless steel, an austenitic stainless steel, an austenitic-ferritic duplex stainless steel, or a precipitation hardened stainless steel, a superelastic alloy such as Ni—Ti alloy, or a radiopaque metal, such as platinum, gold, or tungsten.

In the guidewire according to embodiments of the present invention, the distal portion of the guidewire may be shaped such that a bent portion that is bent at a predetermined angle is formed at a position separated from the front end portion of the front-end solder part toward the rear end portion 13 of the core shaft 10 by a predetermined distance. In the case where the guidewire is shaped, vascular selectivity can be increased and the guidewire can be precisely moved in the lesion. Thus, a guidewire having a high maneuverability can be provided. The shaping angle at which the bent portion is bent is preferably in the range of 10° to 45°, and the distance between the front-end solder part and the bent portion is preferably in the range of 0.5 mm to 5 mm. In this case, vascular selectivity for thin blood vessels and maneuverability in a lesion can be further increased.

The outer surface of the guidewire according to the present invention may be covered with a hydrophilic material. In such a case, the guidewire can be smoothly moved through a guiding catheter, a tube, or a body tissue by reducing the sliding friction.

Examples of the hydrophilic material include cellulose-based high-polymer materials, polyethylene-oxide-based high-polymer materials, maleic-anhydride-based high-polymer materials (e.g., maleic anhydride copolymers such as methyl vinyl ether-maleic anhydride copolymers), acrylamide-based high-polymer materials (e.g., polyacrylamides and polyglycidyl methacrylate-dimethylacrylamide (PGMA-DMAA) block copolymers), water-soluble nylons, polyvinyl alcohols, polyvinyl pyrrolidones, and hyaluronates. In particular, hyaluronates are preferably used.

In the guidewire according to embodiments of the present invention, a connector portion to which an extension guidewire can be attached may be provided at the rear end portion 13 of the core shaft 10.

While the disclosed embodiments have been shown and described in detail, the foregoing description is in all aspects illustrative and not restrictive. Therefore, it is understood that numerous modifications and variations may be devised without departing from the spirit and scope of the invention.

What is claimed is:

1. A guidewire comprising:
a core shaft including
a front section having a front end portion, and
a rear section having a rear end portion;
an outer coil body that covers the front section; and
an inner coil body that is disposed within the outer coil body between the outer coil body and the core shaft, the inner coil body covering the front section, wherein the outer coil body includes
a cylindrical large-diameter section positioned adjacent the rear end portion,
a cylindrical small-diameter section positioned adjacent the front end portion, and
a tapered section that couples the cylindrical large-diameter section to the cylindrical small-diameter section, the tapered section having a diameter that decreases from an end adjacent the rear end portion to an end adjacent the front end portion, and wherein,
the outer coil body includes a first connecting portion that connects the cylindrical large-diameter section to the tapered section and a second connecting portion that connects the cylindrical small-diameter section to the tapered section, and
the inner coil body is disposed within the tapered section and the first connecting portion.

2. The guidewire according to claim 1, wherein
the inner coil body is disposed within the tapered section and both the first connecting portion and the second connecting portion.

3. The guidewire according to claim 1, wherein
the inner coil body is disposed within the tapered section, the second connecting portion, and the cylindrical small-diameter section.

4. The guidewire according to claim 3, wherein
the cylindrical small-diameter section is positioned at a front end of the outer coil body.

5. The guidewire according to claim 4, wherein
the front end portion of the core shaft, a front end portion of the cylindrical small-diameter section, and a front end portion of the inner coil body are coupled to each other at a front tip portion.

6. The guidewire according to claim 5, wherein
the front tip portion has a conical shape.

7. The guidewire according to claim 6, wherein
the front tip portion comprises a solder material containing Au.

8. The guidewire according to claim 7, wherein
the inner coil body is formed of a multiple-wire coil having a plurality of wound wires.

9. The guidewire according to claim 8, wherein
the inner coil body has a tubular shape with a through hole therein.

10. The guidewire according to claim 8, wherein
the multiple-wire coil includes a number of wires that range between about 6 and about 8.

11. The guidewire according to claim 1, wherein
an imaginary line running through a center of the core shaft, along a length of the core shaft, defines a central axis of the guidewire, and
the inner coil body is disposed substantially parallel to the central axis of the guidewire.

12. The guidewire according to claim 1, wherein
the core shaft includes at least two tapering sections, each of the at least two tapering sections having an outer diameter that gradually decreases from a first end to a second end, the at least two tapering sections being interposed by an elongated section having a substantially constant outer diameter.

13. The guidewire according to claim 1, further comprising:
a solder part that fixes the outer coil body to the inner coil body in the first connecting portion.
